(19) United States
(12) Patent Application Publication
(10) Pub. No.: US 2021/0089358 A1
(43) Pub. Date: Mar. 25, 2021

(54) TECHNIQUES FOR IMPROVING PROCESSING OF BIOINFORMATICS INFORMATION TO DECREASE PROCESSING TIME

(71) Applicant: University of Washington, Seattle, WA (US)

(72) Inventors: Ka Yee Yeung-Rhee, Seattle, WA (US); Ling-Hong Hung, Seattle, WA (US); Wesley J. Lloyd, Seattle, WA (US)

(73) Assignee: University of Washington, Seattle, WA (US)

(21) Appl. No.: 17/018,709

(22) Filed: Sep. 11, 2020

Related U.S. Application Data
(60) Provisional application No. 62/903,290, filed on Sep. 20, 2019.

(51) Int. Cl. G06F 9/50; G06F 9/455; G06F 21/64; G16B 25/10

(52) U.S. Cl. CPC G06F 9/5072; G06F 9/45558; G06F 2009/45562; G16B 25/10; G06F 21/64

(57) ABSTRACT

In some embodiments, novel approaches to processing bioinformatics data are used wherein the input data is divided into many small pieces that are processed in parallel by serverless functions. In effect, the functions can be used as on-demand compute units to form an affordable public-cloud version of a supercomputer. Some embodiments of the present disclosure can be used to accelerate the alignment of a single dataset. In some embodiments, the process of setting up the cloud infrastructure is automated so that the user need only to enter credentials from the cloud account. In some embodiments, these improvements are incorporated into an accessible and containerized graphical front-end that allows the user to use a browser to execute, monitor and modify all steps of the analyses from alignment to the display of the resulting differentially expressed genes.

### CROSS-REFERENCE(S) TO RELATED APPLICATION(S)

This application claims the benefit of Provisional Application No. 62/903,290, filed Sep. 20, 2019, the entire disclosure of which is hereby incorporated by reference herein for all purposes.

### STATEMENT OF GOVERNMENT LICENSE RIGHTS

This invention was made with government support under Grant No. R01 GM126019, awarded by the National Institutes of Health. The government has certain rights in the invention.

### BACKGROUND

Biomedical workflows for analyses of genomic data typically consist of many autonomous modules. In the case of RNA-sequencing (RNA-seq), the most computationally intensive step is the mapping of reads to the reference genome. Many optimized algorithms and software tools have been developed for this alignment step. However, the time required for alignment remains substantial. Consequently, once data is aligned, the analyses is rarely repeated even though parameter choices can greatly influence the quality of the alignment.

Many RNA-seq data processing workflows assume that the alignment step has been completed and start with the aligned BAM files or transcript counts tables to compute differential expression. But in fact, the alignment step can be improved with optimized parameters and the entire workflow should be represented as an interactive process with parameter tuning, data wrangling and visualization to facilitate re-analyses so as to improve the quality of the final analytical results.

Cloud computing has been used to accelerate the alignment process by using many cloud-based virtual severs to simultaneously process different datasets. While this approach is effective for rapid processing of large collections of samples, it does not improve the execution time for aligning an individual dataset. Other approaches that divide a single dataset into small pieces to be processed in parallel and/or accelerate specific parts of the alignment algorithm require specialized hardware or maintaining a private or cloud cluster for significant speedup. Recently, function-as-a-service (FaaS) platforms (including but not limited to Amazon Web Services (AWS) Lambda, Google Cloud Functions, IBM Cloud Functions, and Microsoft Azure functions) provide on-demand access to small short-lived applications without the need to configure virtual servers before computation can proceed.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a computer-implemented method for processing bioinformatics data is provided. A first computing system obtains a set of sequence read data. The first computing system divides the set of sequence read data into a set of shards. The first computing system transmits each shard of the set of shards to a serverless function executed by a second computing system. The first computing system gathers intermediate results generated by the serverless function. The first computing system processes the intermediate results to determine final results.

In some embodiments, a system comprising a cloud storage system, a serverless function system, and a management computing system is provided. The management computing system is configured to transmit a set of shards to the cloud storage system; cause the serverless function system to instantiate a number of serverless function computing devices; retrieve the intermediate results from the cloud storage system; and process the intermediate results to determine final results.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram that illustrates a high-level depiction of a data flow and architecture used in non-limiting example embodiments of the present disclosure.

FIG. 2 is a block diagram that illustrates a non-limiting example embodiment of a system according to various aspects of the present disclosure.

FIG. 3A-FIG. 3B are a flowchart that illustrates a non-limiting example embodiment of a method of highly parallel processing of bioinformatics information according to various aspects of the present disclosure.

FIG. 4 is a flowchart that illustrates a non-limiting example embodiment of a procedure for executing a serverless function against an assigned shard according to various aspects of the present disclosure.

FIG. 5 is a flowchart that illustrates a non-limiting example embodiment of a procedure for processing result shards to generate a result according to various aspects of the present disclosure.

FIG. 6 illustrates a non-limiting example embodiment of a graphical user interface (GUI) generated by the GUI engine 226 according to various aspects of the present disclosure.

FIG. 7 is a block diagram that illustrates a non-limiting example embodiment of a computing device appropriate for use as a computing device with embodiments of the present disclosure.

### DETAILED DESCRIPTION

In some embodiments of the present disclosure, novel approaches to processing bioinformatics data are used wherein the input data is divided into many small pieces that are processed in parallel by serverless functions. In effect, the functions can be used as on-demand compute units to form an affordable public-cloud version of a supercomputer. Unlike the approaches designed for processing large collections of samples, some embodiments of the present disclosure can be used to accelerate the alignment of a single dataset. In a non-limiting example embodiment, by invoking 1752 Amazon Web Services® (AWS®) Lambda function instances, alignment time for a 640 million read dataset was reduced from 19.5 hours to 1 minute.

In addition, some embodiments of the present disclosure automate the process of setting up the cloud infrastructure so that the user need only to enter credentials from the cloud account. These improvements are incorporated into an accessible and containerized graphical front-end that allows the user to use a browser to execute, monitor and modify all steps of the analyses from alignment to the display of the resulting differentially expressed genes. The 1100× performance speedup in the alignment coupled with the ease of interactively customizing every step of the analytical pipeline from alignment to visualization has the potential to transform the use of cloud computing in RNA-seq data analyses.

Our case study is an RNA-seq workflow using unique molecular identifiers (UMI) to obtain de-duped transcript counts. This dataset consists of 640 million reads (46 GB compressed files) from cell lines treated with drugs for 48 hours on 96 well plates and was further analyzed using edgeR to obtain a lists of genes that were differentially expressed upon drug treatment. The sequences contain a barcode sequence to identify the sample which allows for multiple samples to be loaded onto the same sequencing lane (multiplexed). After sequencing, the resulting fastq files of reads are processed using a 3-step pipeline to obtain the transcript counts. The first step is a split step that uses the barcode sequences to separate or demultiplex the reads into the 96 originating wells. These reads are then aligned to the human transcriptome using the Burrows-Wheeler Aligner (BWA). The resulting alignments are merged and de-duped using Unique Molecular Identifiers (UMIs) to compute the observed counts for each transcript. The original workflow took 29.5 hours to obtain counts on a cloud server (AWS m4.4×large). The longest step is the CPU-intensive alignment step, which takes 19.5 hours in the original implementation.

In some embodiments of the present disclosure, data splitting is added to the initial de-multiplexing stage that can be performed on a laptop, local server, or cloud server. The resulting small datasets are uploaded to a cloud storage location and serverless functions are invoked to align each dataset in parallel. In some embodiments, the serverless functions combine the alignments and UMI's into a 64-bit hash value which takes 50-fold less space than a compressed SAM file, reducing the time needed to return the result to the cloud bucket. The hash values are then transmitted to the laptop/server which de-dupes and recovers the alignment to generate the final transcript counts. In the case study, the average time for a serverless function instance to align a small fastq file is 39 seconds and the entire alignment process uses 1752 serverless function instances and takes 1 minute (˜1100× faster than the original implementation).

With the elimination of the alignment phase as the bottleneck, the rate limiting steps in the workflow become the data transfer, data splitting and merging steps. Our results show that the entire workflow including the data transfer, split, align, and merge steps to compute the transcript counts can be obtained in approximately 6 minutes at a server operation cost of $3.85. This execution time includes the time needed for data transfer to the cloud server and cost includes the cost of the server.

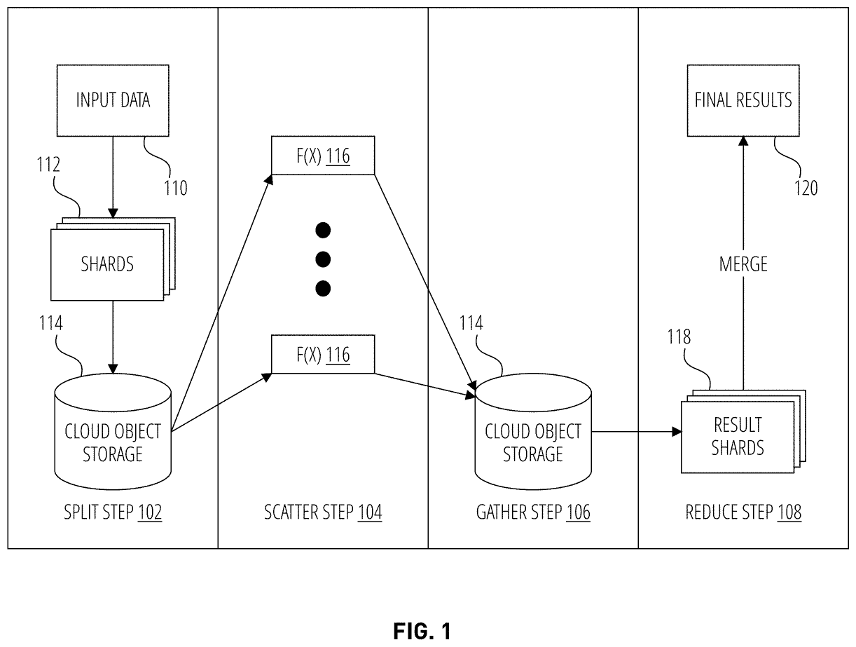

FIG. 1 is a schematic diagram that illustrates a high-level depiction of a data flow and architecture used in non-limiting example embodiments of the present disclosure. As shown, the workflow includes four parts: a split step 102, a scatter step 104, a gather step 106, and a reduce step 108.

In the split step 102, a management computing system receives input data 110, and splits the data into a plurality of shards 112. As discussed below, the size and number of the shards 112 may be chosen by the management computing system in order to maximize the performance gains available from using the workflow. The management computing system transmits the shards 112 to cloud object storage 114, which may be implemented as an S3 bucket or using any other suitable cloud storage system.

In the scatter step 104, the management computing system instantiates a plurality of serverless functions 116, and assigns a shard 112 to each of the serverless functions 116. Each serverless function 116 then retrieves its shard 112 from cloud object storage 114 and conducts its processing. In the gather step 106, each serverless function 116 stores its result back in cloud object storage 114. Because the nature of cloud object storage 114 makes it capable of highly efficient parallel communication with the serverless functions 116, the parallel operation of the plurality of serverless functions 116 in retrieving, processing, and storing results back in the cloud object storage 114 leads to massive acceleration of the overall workflow.

Finally, in the reduce step 108, the management computing system retrieves result shards 118 from cloud object storage 114, and merges the result shards 118 to obtain final results 120. Further description of each of these actions is provided below.

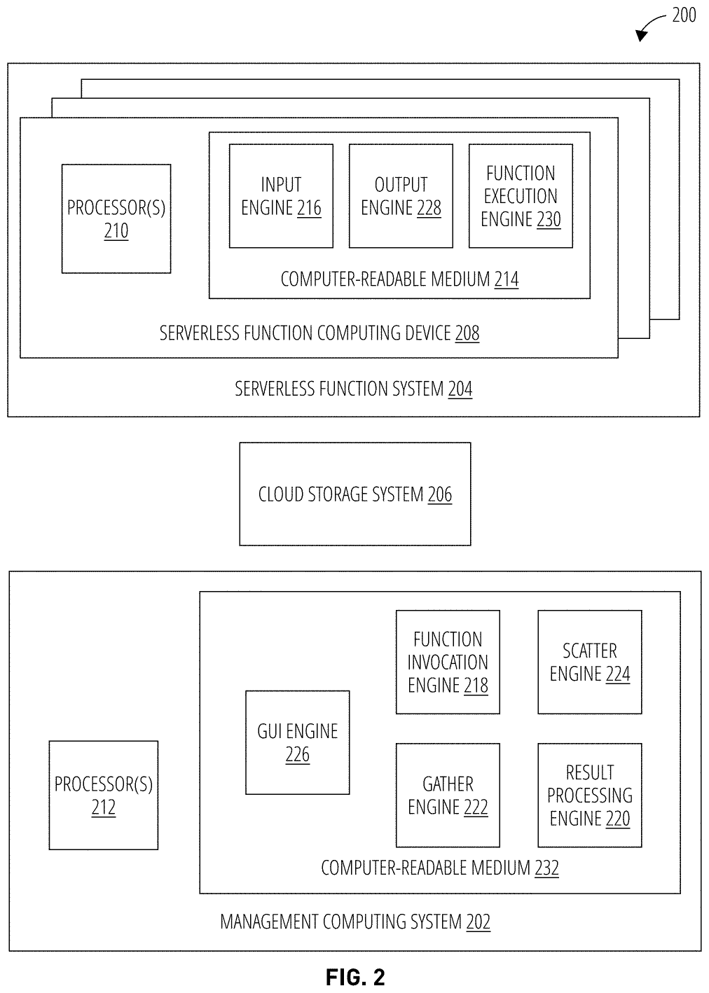

FIG. 2 is a block diagram that illustrates a non-limiting example embodiment of a system according to various aspects of the present disclosure. The illustrated system 200 is a non-limiting example of a system suitable for implementing the workflow illustrated in FIG. 1.

As shown, the system includes a management computing system 202, a serverless function system 204, and a cloud storage system 206.

In some embodiments, the management computing system 202 is any type of computing device or collection of computing devices configured to provide the described functionality. In some embodiments, the management computing system 202 includes a laptop computing device, a desktop computing device, a rack-mount server computing device, a mobile computing device (including but not limited to a smartphone or a tablet computing device), one or more computing devices in a cloud computing system, and/or any other type of computing device or combination of computing devices. In some embodiments, the elements illustrated present on the computer-readable medium 232 may be packaged in a container of a type including but not limited to a Docker container, such that many different types of computing devices may provide the functionality of the management computing system 202 by executing the container in a virtual machine. In some embodiments, a cloud computing provider that is paired with the cloud storage system 206 may be selected to run the management computing system 202, such that the data transfer time between the management computing system 202 and the cloud storage system 206 is minimized.

As shown, the management computing system 202 includes one or more processor(s) 212 and a computer-readable medium 232. In some embodiments, the processor(s) 212 are typical general purpose computing processors. In some embodiments, the processor(s) 212 may include one or more specialized computing processors, including but not limited to processors optimized for graphical computations (GPUs) and/or processors optimized for machine learning, security, networking communication, or other tasks. In some embodiments, the computer-readable medium 232 may be any suitable type of non-transitory computer-readable medium, including but not limited to one or more hard drives, one or more flash memory devices, and/or one or more optical storage devices.

As shown, the computer-readable medium 232 includes a GUI engine 226, a function invocation engine 218, a gather engine 222, a scatter engine 224, and a result processing engine 220. The specific illustration and description of the engines shown in FIG. 2 is provided for the sake of clarity. In some embodiments, the functionality of two or more engines illustrated in FIG. 2 may be combined into a single engine, while in some embodiments, the functionality of a single engine illustrated in FIG. 2 may be separated into multiple separate engines without departing from the scope of the present disclosure.

In some embodiments, the GUI engine 226 is configured to cause a graphical user interface (GUI) to be presented to a user. In some embodiments, the GUI engine 226 may cause the GUI to be presented on a display device of the management computing system 202. In some embodiments, the GUI engine 226 may operate a web server or other type of information service that allows the GUI to be presented on a display device of a computing device other than the management computing system 202. In some embodiments, the GUI both presents information about the workflow (including but not limited to data flow paths, configurations, and results) and accepts input from a user to cause changes to be made in the configuration of the workflow.

In some embodiments, the scatter engine 224 is configured to divide input data 110 into a plurality of shards 112, and to transfer the plurality of shards 112 to the cloud storage system 206. In some embodiments, the function invocation engine 218 is configured to cause each of the serverless functions 116 to process its associated shard 112. In some embodiments, the gather engine 222 is configured to retrieve result shards 118 from the cloud storage system 206. In some embodiments, the result processing engine 220 is configured to process the result shards 118 to create final results 120. Further description of each of these tasks is provided below.

In some embodiments, the serverless function system 204 is a cloud computing system that provides on-demand computing services. In the serverless function system 204, the cloud computing system dynamically manages the allocation of hardware computing devices to provide the illustrated components. Typically, the serverless function system 204 charges a user for the amount of computing resources actually consumed by the components, as opposed to charging for the guaranteed availability of particular computing devices. Some non-limiting examples of services capable of providing a serverless function system 204 include Amazon Web Services (AWS) Lambda, Google® Cloud Functions, IBM® Cloud Functions, and Microsoft® Azure functions. In some embodiments, the serverless function system 204 may be provided by one of these public services. In some embodiments, the serverless function system 204 may be provided by a private service, or could run on a private cluster of computing devices.

As shown, the serverless function system 204 includes a plurality of serverless function computing devices 208. In some embodiments, each serverless function computing device 208 represents a computing device allocated by the serverless function system 204 to provide the illustrated components. In some embodiments, a user may specify capabilities of the serverless function computing device 208 to be allocated by the serverless function system 204, including but not limited to one or more of a maximum processing speed, an amount of available local storage, and an available network bandwidth. In some embodiments, the management computing system 202 instructs the serverless function system 204 to instantiate the plurality of serverless function computing devices 208, provides the desired capabilities of the serverless function computing devices 208, and provides the serverless function system 204 with the components to be stored on the computer-readable medium 214 of each serverless function computing device 208 as described below.

As shown, each serverless function computing device 208 includes one or more processor(s) 210 and a computer-readable medium 214. In some embodiments, the processor(s) 210 are typical general purpose computing processors. In some embodiments, the processor(s) 210 may include one or more specialized computing processors, including but not limited to processors optimized for graphical computations (GPUs) and/or processors optimized for machine learning, security, networking communication, or other tasks. In some embodiments, the computer-readable medium 214 may be any suitable type of non-transitory computer-readable medium, including but not limited to one or more hard drives, one or more flash memory devices, and/or one or more optical storage devices.

As shown, the computer-readable medium 214 includes an input engine 216, an output engine 228, and a function execution engine 230. In some embodiments, the input engine 216 is configured to retrieve an assigned shard 112 from the cloud storage system 206. In some embodiments, the output engine 228 is configured to store a result shard 118 in the cloud storage system 206. In some embodiments, the function execution engine 230 is configured to perform processing on data within the shard 112 to create the result shard 118. Further description of the actions performed by each of these components is provided below.

In some embodiments, the cloud storage system 206 includes a plurality of computing devices configured to provide high-capacity storage of information, including but not limited to the cloud object storage 114 illustrated in FIG. 1. Typically, the cloud storage system 206 includes geographically distributed computing devices that provide massively parallel and redundant storage. In some embodiments, any commercially available cloud storage system 206 may be used. In some embodiments, a cloud storage system 206 that is paired with the serverless function system 204 may be used in order to obtain the most efficient transmission of data between the cloud storage system 206 and the serverless function computing devices 208. For example, if the serverless function system 204 used is Amazon Web Services Lambda, then a suitable cloud storage system 206 may be Amazon Simple Storage Service (S3). Because more than one cloud storage system 206 is commercially available and known to those of ordinary skill in the art, the details of the cloud storage system 206 are not descried further herein for the sake of brevity. In some embodiments, the cloud storage system 206 may be provided by one of these public services. In some embodiments, the cloud storage system 206 may be provided by a private service, or could run on a private cluster of computing devices.

As used herein, “engine” refers to logic embodied in hardware or software instructions, which can be written in one or more programming languages, including but not limited to C, C++, C#, COBOL, JAVA™, PHP, Perl, HTML, CSS, JavaScript, VBScript, ASPX, Go, and Python. An engine may be compiled into executable programs or written in interpreted programming languages. Software engines may be callable from other engines or from themselves. Generally, the engines described herein refer to logical modules that can be merged with other engines, or can be divided into sub-engines. The engines can be implemented by logic stored in any type of computer-readable medium or computer storage device and be stored on and executed by one or more general purpose computers, thus creating a special purpose computer configured to provide the engine or the functionality thereof. The engines can be implemented by logic programmed into an application-specific integrated circuit (ASIC), a field-programmable gate array (FPGA), or another hardware device, though typically, if designed to be transmitted across a network and executed by a remote computing device (such as the engines of the serverless function computing device 208), the engines will be implemented as computer-executable instructions stored on a computer-readable medium instead of as an ASIC, an FPGA, or another hardware device.

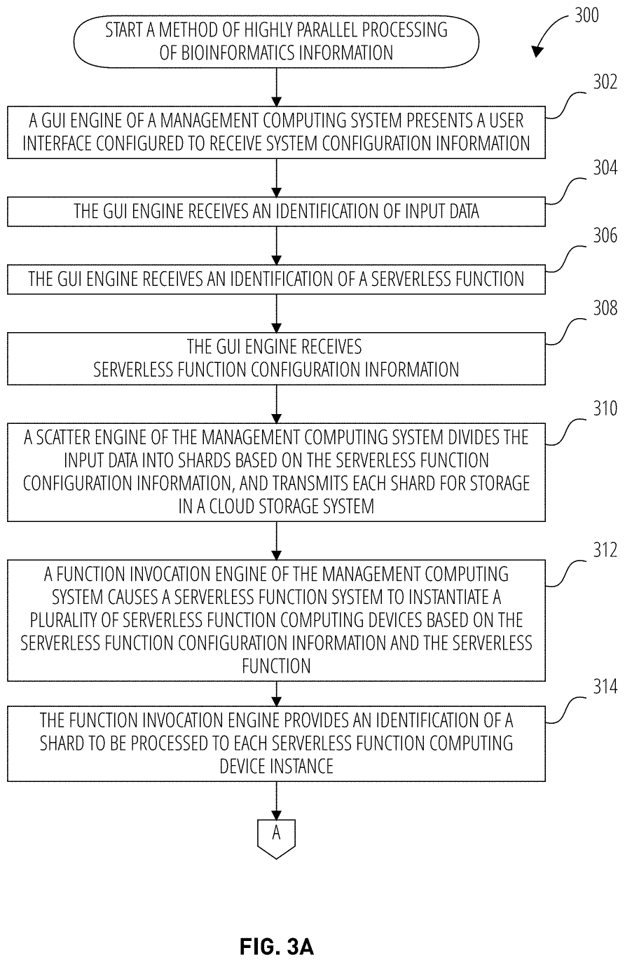

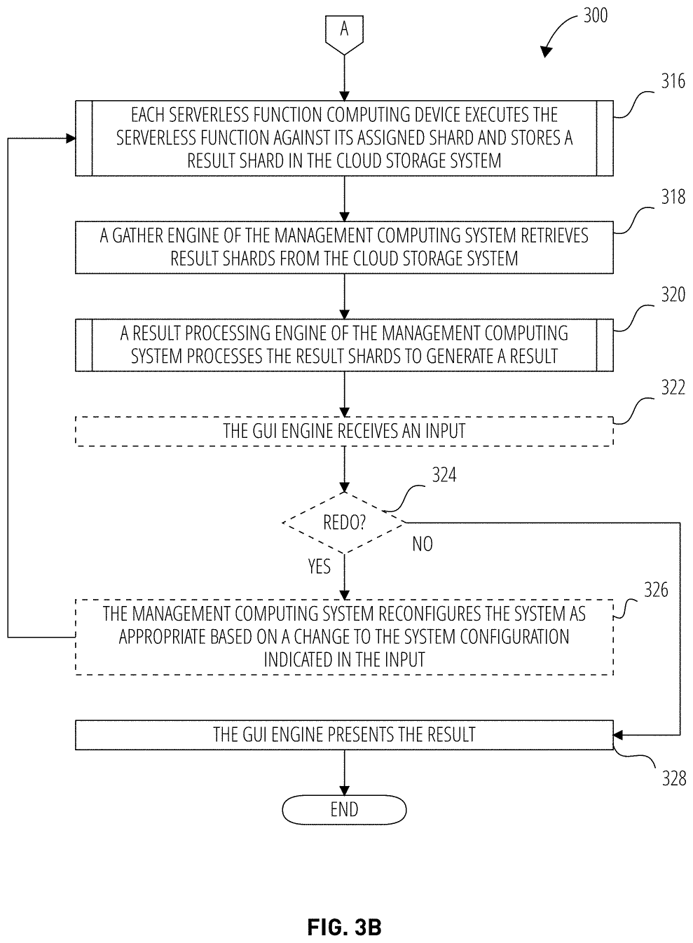

FIG. 3A-FIG. 3B are a flowchart that illustrates a non-limiting example embodiment of a method of highly parallel processing of bioinformatics information according to various aspects of the present disclosure. The method 300 is a non-limiting example of a detailed description of the high-level workflow illustrated in FIG. 1.

From a start block, the method 300 advances to block 302, where a GUI engine 226 of a management computing system 202 presents a user interface configured to receive system configuration information. In some embodiments, the user interface may be presented on a display device of the management computing system 202. In some embodiments, the user interface may be provided by a web server or other programmatically accessible interface executed by the management computing system 202, and may be presented by a display device of a computing device separate from the management computing system 202.

At block 304, the GUI engine 226 receives an identification of input data. In some embodiments, the input data may include raw sequencing data such as RNA-seq data, and may be stored in any suitable format, including but not limited to fastq format. In some embodiments, the identification of the input data may be a file name, an object identifier, a URI, or any other suitable identification of a storage location of the input data. In some embodiments, upon receiving the identification of the input data, the management computing system 202 may copy the input data from a location associated with the identification (such as a location in the cloud storage system 206) to a local storage location that may be manipulated by components of the management computing system 202.

At block 306, the GUI engine 226 receives an identification of a serverless function 116. In some embodiments, the GUI engine 226 may provide a list of available serverless functions 116, and the identification may be a selection of one of the available serverless functions 116 by a user. In some embodiments, the serverless functions 116 may include logic for processing a portion of the input data to create a portion of a result. In many bioinformatics workflows, the logic for each serverless function 116 may implement a particular algorithm, such as an alignment algorithm that generates alignments to a transcriptome or reference genome for sequence reads in the input data. Some non-limiting examples of algorithms that may be implemented by the serverless functions 116 include a Burrows-Wheeler Aligner (BWA), Smith-Waterman alignments, Striped Smith-Waterman (SSW) alignments, and rapid pseudo-alignment techniques such as that used in the kallisto-sleuth RNA-seq data processing pipeline.

At block 308, the GUI engine 226 receives serverless function configuration information. In some embodiments, the serverless function configuration information includes one or more configuration settings for the particular technique implemented by the identified serverless function 116. In some embodiments, the serverless function configuration information may include one or more pre-processing or post-processing utilities to be installed on the serverless function system 204 to support execution of the serverless function 116. In some embodiments, the serverless function configuration information may include a set of reference data, including but not limited to a reference genome or transcriptome, to be used by the serverless function 116.

In some embodiments, the serverless function configuration information may include one or more indications of a target for an output of a processing step and/or a source for an input of a processing step. For example, if an algorithm implemented by the serverless function 116 includes more than one interchangeable step, the serverless function configuration information may identify each interchangeable step, and may “wire up” specific outputs of one step to specific inputs of another step.

In some embodiments, the serverless function configuration information may include one or more desired capabilities of the serverless function computing device 208 to be instantiated to execute the serverless function 116. For example, the serverless function configuration information may include a desired amount of storage, a desired amount of memory, a desired amount of network bandwidth, and/or a desired processing power for each serverless function computing device 208. In some embodiments, the desired amounts of storage, memory, network bandwidth, and/or processing power may be determined based on prices charged by the provider of the serverless function system 204 to instantiate such serverless function computing devices 208, such that the desired amounts may be chosen to be below thresholds that would increase the cost charged by the provider of the serverless function system 204 to execute the workflow.

In some embodiments, the serverless function configuration information may include a number of serverless function computing devices 208 to be instantiated. In some embodiments, the number of serverless function computing devices 208 instantiated is selected to maximize the throughput of the overall workflow. Because the largest bottleneck in bioinformatics workflows is generally the amount of time that it takes to execute the algorithms that are executed in the method 300 within the serverless function 116, and because retrieval of data by the serverless function computing devices 208 from the cloud storage system 206 can be performed efficiently in a massively parallel manner, it may be beneficial to instantiate a large number of serverless function computing devices 208 to take maximum advantage of the efficiency of the parallel processing. To obtain maximum advantage, the number of serverless function computing devices 208 used may, counterintuitively, be even greater than a minimum number of serverless function computing devices 208 for storing the input data 110 (that is, each of the serverless function computing devices 208 may have excess storage space or computing power compared to that strictly needed to support processing of a shard 112). This is counterintuitive at least because a traditional technique would attempt to use the fewest computing devices and/or computing devices of a minimum suitable computing power. It has been found that by using the techniques disclosed herein (including, but not limited to, the use of a serverless function system 204 to manage the distributed result computing), the minimal additional computing costs of using more parallel computing devices are more than outweighed by the speed and efficiency benefits gained.

For instance, in one non-limiting example workflow, 1752 serverless function computing devices 208 were selected to be instantiated to process a 48 GB input data set. In some embodiments, more or fewer serverless function computing devices 208 may be selected to be instantiated. Typically, the number of serverless function computing devices 208 to be instantiated may be determined based on a tradeoff of the increased processing power provided by the additional instances versus an amount of time that it takes the serverless function system 204 to instantiate the instances. In some embodiments, a minimum number of serverless function computing devices 208 may be determined based on a maximum storage size allotted to a single serverless function computing device 208 and an overall size of the input data 110.

At block 310, a scatter engine 224 of the management computing system 202 divides the input data 110 into shards 112 based on the serverless function configuration information, and transmits each shard 112 for storage in a cloud storage system 206. In some embodiments, one shard 112 is created for each serverless function computing device 208, and the input data 110 is divided equally between the shards 112.

At block 312, a function invocation engine 218 of the management computing system 202 causes a serverless function system 204 to instantiate a plurality of serverless function computing devices 208 based on the serverless function configuration information and the serverless function 116. In some embodiments, this includes creating a container or other package that contains the logic of the engines of the serverless function computing device 208, as well as any other supporting data or executables used by the engines, and transmitting the container to the serverless function system 204. In some embodiments, this also includes instructing the serverless function system 204 regarding the desired capabilities of each serverless function computing device 208 to instantiate, and the number of serverless function computing devices 208 to instantiate. In response, the serverless function system 204 will begin instantiating the serverless function computing devices 208 as instructed.

At block 314, the function invocation engine 218 provides an identification of a shard 112 to be processed to each serverless function computing device 208 instance. In some embodiments, the function invocation engine 218 may provide the identification of each shard 112, once it has completed transfer to the cloud storage system 206, to a message queue of the serverless function system 204. Once each serverless function computing device 208 has completed its startup, it subscribes to the message queue. Accordingly, a configured serverless function computing device 208 will pick up the message from the message queue to receive the identification of its shard 112.

The method 300 then advances to a continuation terminal (“terminal A”). From terminal A (FIG. 3B), the method 300 proceeds to subroutine block 316, where a procedure is executed wherein each serverless function computing device 208 executes the serverless function 116 against its assigned shard 112 and stores a result shard 118 in the cloud storage system 206.

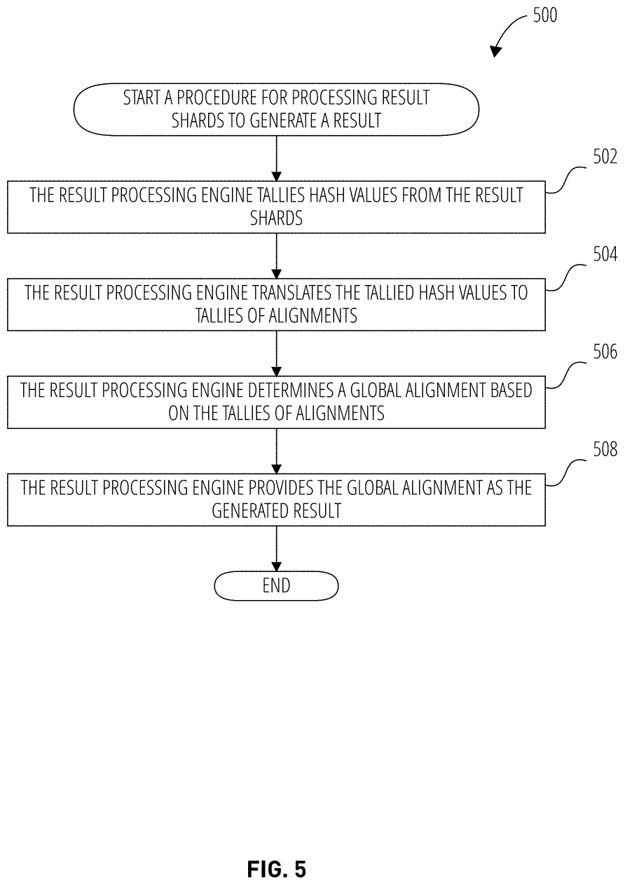

At block 318, a gather engine 222 of the management computing system 202 retrieves result shards 118 from the cloud storage system 206. The method 300 then advances to subroutine block 320, where a procedure is executed wherein a result processing engine 220 of the management computing system 202 processes the result shards 118 to generate a result. Any suitable technique that corresponds to the technique used by the serverless function 116 may be used to generate the result at subroutine block 320, including but not limited to the procedure 500 illustrated in FIG. 5 and discussed in detail below.

In some embodiments, the increases in the speed of the calculation of the result by the method 300 are so great that they allow the user interface to be used in an essentially interactive manner. Since the results can be generated by the method 300 on a time scale ranging from seconds to a small number of minutes, it becomes feasible for the user to change settings and cause the analysis to be re-run after viewing a preliminary result. Accordingly, at optional block 322, the GUI engine 226 receives an input. Though illustrated as occurring after subroutine block 320, the input received by the GUI engine 226 at optional block 322 may be received at any preceding point of the method 300, including but not limited to during the processing of subroutine block 316 or subroutine block 320.

The method 300 then advances to optional decision block 324, where a determination is made regarding whether the calculations of the serverless functions 116 should be redone based on the input. In some embodiments, the input may indicate an additional visualization or other post-alignment processing to be done on the result, in which case the calculations of the serverless function 116 may not need to be redone. In some embodiments, the input may indicate a change to serverless function configuration information that would affect the way that the serverless functions 116 operate, including but not limited to replacing an alignment technique used by the serverless functions 116 and changing a configuration setting for the serverless functions 116, in which case the calculations of the serverless functions 116 should be redone.

If the calculations should be redone, then the result of optional decision block 324 is YES, and the method 300 proceeds to optional block 326, where the management computing system 202 reconfigures the system 200 as appropriate based on a change to the system configuration indicated in the input. The reconfiguration performed depends on the input, but may be any configuration of the system 200 as discussed above, including but not limited to adding, removing, reconfiguring, and/or reordering one or more processing steps. The method 300 then returns to subroutine block 316 to perform the calculations again. In some embodiments, the method 300 may return to an earlier portion of the method 300 if the input indicated changes in a configuration made before subroutine block 316.

If the calculations should not be redone, then the method 300 proceeds to block 328. One will note that optional block 322-optional block 326 are illustrated as optional because in some embodiments, the method 300 may not receive an input after the result is generated by the management computing system 202. In such embodiments, the method 300 proceeds directly from subroutine block 320 to block 328.

At block 328, the GUI engine 226 presents the result. In some embodiments, the management computing system 202 may save the result to a computer-readable medium and/or transmit the result to another computing device instead of or in addition to presenting the result. In some embodiments, the input of optional block 322 may be received after the result is displayed, in which case the method 300 would advance from block 328 to optional block 322.

The method 300 then proceeds to an end block and terminates.

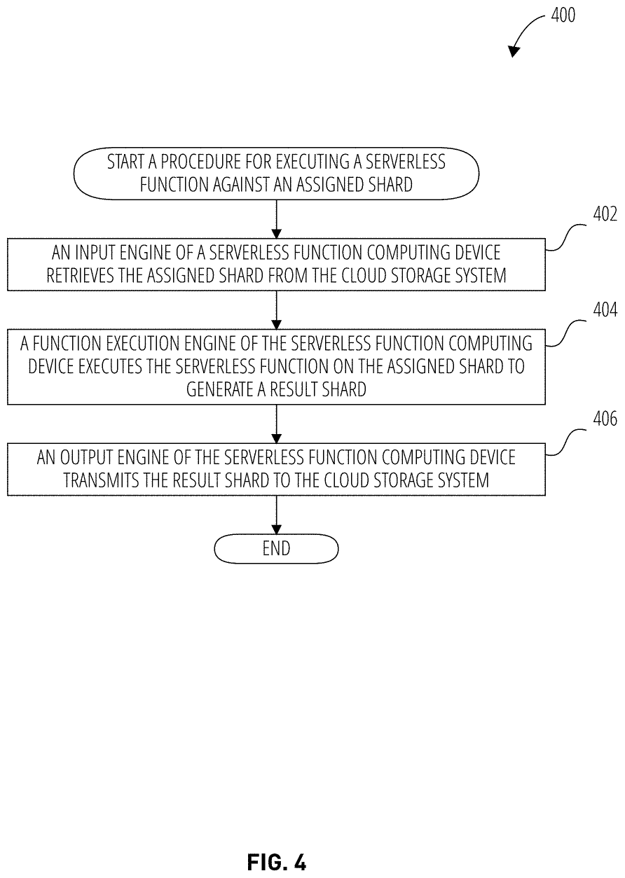

FIG. 4 is a flowchart that illustrates a non-limiting example embodiment of a procedure for executing a serverless function against an assigned shard according to various aspects of the present disclosure. The procedure 400 is a non-limiting example of a procedure suitable for use at subroutine block 316 of method 300 as discussed above. In the procedure 400, the serverless function 116 processes a shard 112 assigned by the scatter engine 224 of the management computing system 202, and stores a result shard 118 in the cloud storage system 206. The procedure 400 assumes that the serverless function computing device 208 has received an assignment of a shard 112 from the management computing system 202.

From a start block, the procedure 400 advances to block 402, where an input engine 216 of a serverless function computing device 208 retrieves the assigned shard 112 from the cloud storage system 206. In some embodiments, this may be performed by retrieving a name of a shard 112 stored in the cloud storage system 206 from a message queue of the serverless function system 204, and then copying the shard 112 from the cloud storage system 206 to local storage of the serverless function computing device 208.

At block 404, a function execution engine 230 of the serverless function computing device 208 executes the serverless function 116 on the assigned shard to generate a result shard 118. In some embodiments, an algorithm (including but not limited to the BWA algorithm and other techniques described above) is executed by the serverless function 116 against its assigned shard 112 to produce one or more alignments, pseudocounts, or other results for the sequence reads in the shard 112.

The standard format to store alignments are text files in the Sequence Alignment Map (SAM) format, or compressed versions such as Binary Alignment Map (BAM) or CRAM of the SAM format. In some embodiments, one of these formats may be used by the serverless function 116 to store the alignments in a result shard 118. In some embodiments, the serverless function 116 is configured to store an even further condensed version of the alignments in order to reduce the amount of data that needs to be transferred between the serverless function 116, the cloud storage system 206, and the management computing system 202. Accordingly, in some embodiments, the serverless function 116 creates a perfect hash value for each alignment that uniquely identifies the position of the alignment and a unique molecular identifier (UMI) sequence. Because a perfect hash function is used, identical hash values are only possible if two reads map to the same position and have the same UMI (i.e., are amplification duplicates), and allows the alignment position and UMI sequences to be recovered by the management computing system 202. These perfect hash values are then stored by the serverless function 116 in the result shard 118. The storing of perfect hash values instead of standard SAM files can reduce the amount of disk storage needed, and reduces the amount of data transferred over the network by at least 50×.

At block 406, an output engine 228 of the serverless function computing device 208 transmits the result shard 118 to the cloud storage system 206. The procedure 400 then advances to an end block and terminates. One will recognize that the procedure 400 is generally executed by a number of serverless function computing devices 208 in parallel to concurrently process multiple different shards 112.

FIG. 5 is a flowchart that illustrates a non-limiting example embodiment of a procedure for processing result shards to generate a result according to various aspects of the present disclosure. The procedure 500 is an example of a procedure suitable for use at subroutine block 320 of method 300 illustrated and discussed above. In the procedure 500, the management computing system 202 combines the information in the result shards 118 to create an overall result of the processing. The procedure 500 assumes that the gather engine 222 has already gathered at least some result shards 118 from the cloud storage system 206. The procedure 500 also assumes that the result shards 118 were generated using a procedure that uses perfect hash values to represent the positions of the alignments and the sequences that are aligned, as described above.

In some embodiments, the procedure 500 may not wait for all of the result shards 118 to be retrieved before starting, but may instead process result shards 118 asynchronously as they are retrieved. It has been found that the asynchronous merging of results from the result shards 118 can reduce the total amount of elapsed time for conducting the merge by 50%.

From a start block, the procedure 500 advances to block 502, where the result processing engine 220 of the management computing system 202 tallies hash values from the result shards 118. In some embodiments, the result processing engine 220 determines which hash values are present in the result shards 118, and discards duplicate hash values in order to obtain a set of unique hash values representing the alignment positions and unique molecular identifiers found.

At block 504, the result processing engine 220 translates the tallied hash values to tallies of alignments. Because a perfect hash function is used, each hash value maps to exactly one combination of an alignment position and UMI. Accordingly, the result processing engine 220 can translate each hash value to the corresponding alignment position and UMI.

At block 506, the result processing engine 220 determines a global alignment based on the tallies of alignments. Any suitable technique for converting the alignment tallies to the global alignment may be used, including but not limited to hashing unique molecular identifying sequences or using multiple reference sequences.

At block 508, the result processing engine 220 provides the global alignment as the result. The procedure 500 then advances to an end block and terminates.

The above description of method 300, procedure 400, and procedure 500 relates primarily to a sequence alignment based on fastq data, because this is an example of a workflow that is particularly amenable to being accelerated by these techniques. However, this example should not be seen as limiting. In some embodiments, other bioinformatics workflows could be processed using a serverless function system 204 in similar ways by providing appropriate logic for the serverless function 116 and the gather engine 222. Other examples of bioinformatics workflows that could be accelerated using these techniques include, but are not limited to, variant discovery using high-throughput sequencing data, protein-folding, deep-learning, data clustering, molecular simulations, cheminformatics, and other algorithms with GPU implementations that could easily be adapted to execute on a cloud storage system 206 instead of a GPU.

Further, one will recognize that although the blocks of method 300, procedure 400, and procedure 500 are illustrated linearly for ease of discussion, many of the blocks may operate in a different order or concurrently. For example, in some embodiments, the scatter engine 224 may store each shard 112 in the cloud storage system 206 when it is ready, instead of waiting for all of the shards 112 to be ready before transmitting. Similarly, in some embodiments, the actions of block 312 may begin before the processing of block 310 is complete so that the serverless function computing devices 208 may start getting instantiated while the shards 112 are being generated and stored, and once instantiation of a given serverless function computing device 208 is complete, it may proceed to block 314 and beyond without waiting for the rest of the serverless function computing devices 208 to be ready.

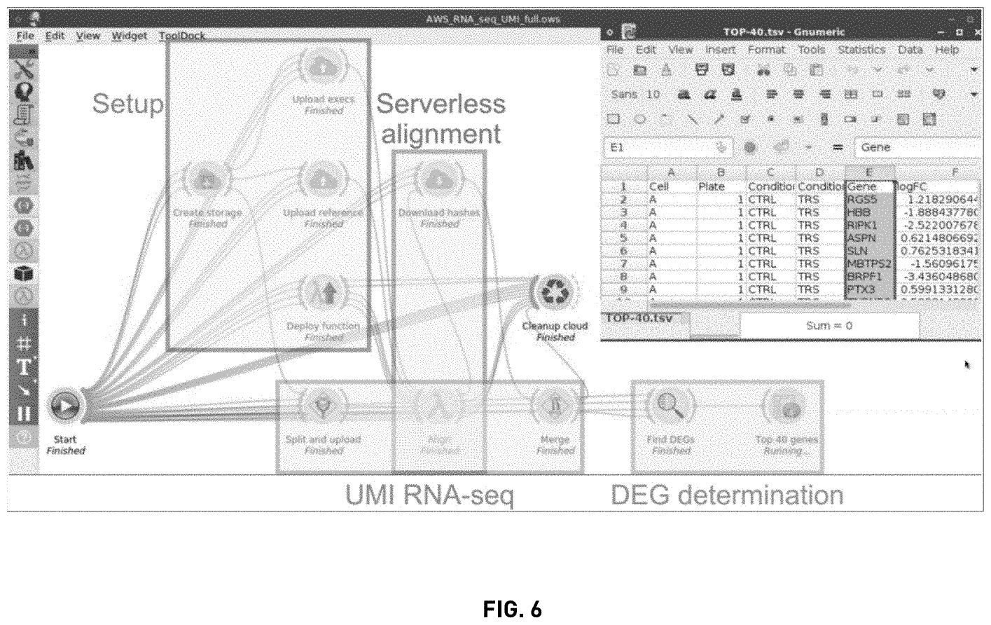

FIG. 6 illustrates a non-limiting example embodiment of a graphical user interface (GUI) generated by the GUI engine 226 according to various aspects of the present disclosure. As shown, the GUI engine 226 may be executing on any type of computing device, and may be accessed by a user via a web browser.

Each circular interface element in the interface provides a control for serverless function configuration information relating to a separate containerized module. The containerized modules may indicate any configurable portion of the system 200, including but not limited to modules for providing/configuring any of the engines or other components of the system 200. The containerized modules may also provide other functionality other than that illustrated and discussed above, including but not limited to various visualizations and/or transformations of the final results 120.

Double-clicking on a given interface element reveals further graphical elements for parameter entry, starting/stopping execution, and/or presenting intermediate output. The curved lines connecting the circular interface elements indicate data flow between the execution modules; a location on the right side of a first interface element connected to a curved line indicates an output value of the module associated with the first interface element, and a location on the left side of a second interface element connected to the curved line indicates an input value of the module associated with the second interface element. Connections and interface elements representing containerized modules can be added and removed from the toolbox on the left side of the GUI using a drag-and-drop interface.

In the illustrated embodiment, the workflow itself is started by double-clicking on the start interface element in the lower left of the window. The user then enters the names of one or more data files that include the input data 110, credentials for the cloud storage system 206, and credentials for the serverless function system 204 into a form, and actuates a start button to begin execution. Execution proceeds automatically and finishes by popping up a fully functional spreadsheet populated with a list of differentially expressed genes. The user can monitor, stop, modify, and restart the workflow at any point. Boxes have been overlaid on the GUI for illustrative purposes to show the grouping of interface elements relevant to setup, alignment using serverless computing, a UMI RNA-seq pipeline, and differentially expressed gene (DEG) determination using edgeR. A cleanup module is also provided to delete cloud resources once the user is finished.

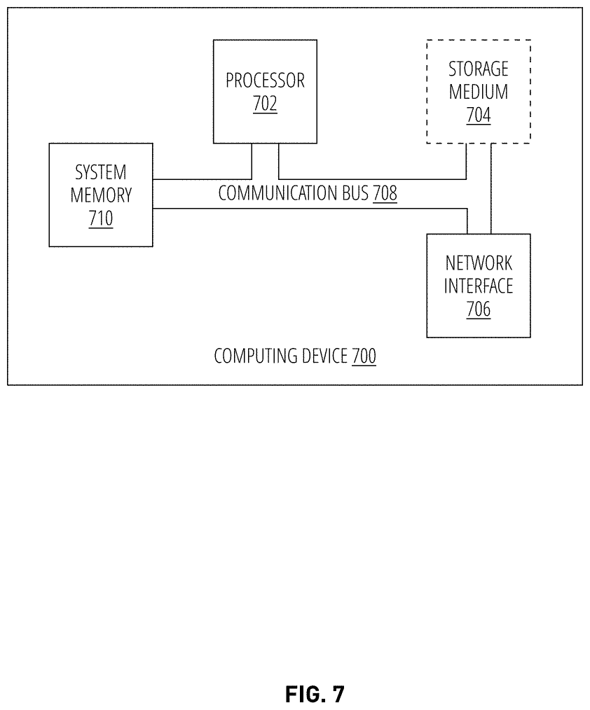

FIG. 7 is a block diagram that illustrates aspects of an exemplary computing device 700 appropriate for use as a computing device of the present disclosure. While multiple different types of computing devices were discussed above, the exemplary computing device 700 describes various elements that are common to many different types of computing devices. While FIG. 7 is described with reference to a computing device that is implemented as a device on a network, the description below is applicable to servers, personal computers, mobile phones, smart phones, tablet computers, embedded computing devices, and other devices that may be used to implement portions of embodiments of the present disclosure. Some embodiments of a computing device may be implemented in or may include an application-specific integrated circuit (ASIC), a field-programmable gate array (FPGA), or other customized device. Moreover, those of ordinary skill in the art and others will recognize that the computing device 700 may be any one of any number of currently available or yet to be developed devices.

In its most basic configuration, the computing device 700 includes at least one processor 702 and a system memory 710 connected by a communication bus 708. Depending on the exact configuration and type of device, the system memory 710 may be volatile or nonvolatile memory, such as read only memory (“ROM”), random access memory (“RAM”), EEPROM, flash memory, or similar memory technology. Those of ordinary skill in the art and others will recognize that system memory 710 typically stores data and/or program modules that are immediately accessible to and/or currently being operated on by the processor 702. In this regard, the processor 702 may serve as a computational center of the computing device 700 by supporting the execution of instructions.

As further illustrated in FIG. 7, the computing device 700 may include a network interface 706 comprising one or more components for communicating with other devices over a network. Embodiments of the present disclosure may access basic services that utilize the network interface 706 to perform communications using common network protocols. The network interface 706 may also include a wireless network interface configured to communicate via one or more wireless communication protocols, such as Wi-Fi, 2G, 3G, LTE, WiMAX, Bluetooth, Bluetooth low energy, and/or the like. As will be appreciated by one of ordinary skill in the art, the network interface 706 illustrated in FIG. 7 may represent one or more wireless interfaces or physical communication interfaces described and illustrated above with respect to particular components of the computing device 700.

In the exemplary embodiment depicted in FIG. 7, the computing device 700 also includes a storage medium 704. However, services may be accessed using a computing device that does not include means for persisting data to a local storage medium. Therefore, the storage medium 704 depicted in FIG. 7 is represented with a dashed line to indicate that the storage medium 704 is optional. In any event, the storage medium 704 may be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

Suitable implementations of computing devices that include a processor 702, system memory 710, communication bus 708, storage medium 704, and network interface 706 are known and commercially available. For ease of illustration and because it is not important for an understanding of the claimed subject matter, FIG. 7 does not show some of the typical components of many computing devices. In this regard, the computing device 700 may include input devices, such as a keyboard, keypad, mouse, microphone, touch input device, touch screen, tablet, and/or the like. Such input devices may be coupled to the computing device 700 by wired or wireless connections including RF, infrared, serial, parallel, Bluetooth, Bluetooth low energy, USB, or other suitable connections protocols using wireless or physical connections. Similarly, the computing device 700 may also include output devices such as a display, speakers, printer, etc. Since these devices are well known in the art, they are not illustrated or described further herein.

While illustrative embodiments have been illustrated and described, it will be appreciated that various changes can be made therein without departing from the spirit and scope of the invention. As one example, though the above description includes much description of a case study regarding the use of serverless functions to accelerate the computation of an alignment step for RNA sequencing, similar techniques may be used to accelerate DNA sequencing or other computations, and may be used to accelerate other steps of the overall process instead of just the alignment step.

The embodiments of the invention in which an exclusive property or privilege is claimed are defined as follows:

1. A computer-implemented method for processing bioinformatics data, the method comprising:
obtaining, by a first computing system, a set of sequence read data;
dividing, by the first computing system, the set of sequence read data into a set of shards;
transmitting, by the first computing system, each shard of the set of shards to a serverless function executed by a second computing system;
gathering, by the first computing system, intermediate results generated by the serverless function; and
processing, by the first computing system, the intermediate results to determine final results.

2. The computer-implemented method of claim 1, wherein dividing the set of sequence read data into a set of shards includes dividing the set of sequence read data into at least a number of subsets of sequence read data sized such that each subset of sequence read data fits within a data size limit of the second computing system.

3. The computer-implemented method of claim 2, wherein dividing the set of sequence read data into a set of shards includes storing the subsets of sequence read data in a cloud storage system.

4. The computer-implemented method of claim 3, wherein transmitting each shard of the set of shards to a serverless function executed by the second computing system includes assigning a subset of the sequence read data stored in the cloud storage system to an instance of a serverless function.

5. The computer-implemented method of claim 1, wherein the intermediate results include perfect hash values that represent at least an alignment.

6. The computer-implemented method of claim 5, wherein processing the intermediate results to determine final results includes counting matching perfect hash values to determine counts of unique sequences.

7. The computer-implemented method of claim 1, wherein the set of sequence read data is a set of RNA sequence reads.

8. The computer-implemented method of claim 1, wherein the final results include gene expression information.

9. The computer-implemented method of claim 1, wherein the intermediate results are pseudocounts, and final results are differential gene expressions.

10. The computer-implemented method of claim 1, further comprising presenting, by the first computing system, a user interface that includes one or more interface elements configured to receive input for configuring actions of the first computing system and the second computing system, wherein the one or more interface elements include one or more of:
an interface element for receiving a number of shards for the set of shards;
an interface element for receiving an identification of the serverless function;
an interface element for receiving an identification of an alignment technique;
an interface element for receiving an indication of a data flow path between containerized modules; and
an interface element for receiving instructions for adding, removing, and reordering processing steps.

11. The computer-implemented method of claim 10, wherein the interface element for adding, removing, and reordering processing steps is configured to receive an instruction for at least one of adding, removing, and reordering a processing step after the set of sequence read data is obtained and before the final results are determined.

12. A system, comprising:
a cloud storage system;
a serverless function system; and
a management computing system, wherein the management computing system is configured to:
transmit a set of shards to the cloud storage system;
cause the serverless function system to instantiate a number of serverless function computing devices to generate intermediate results;
retrieve the intermediate results from the cloud storage system; and
process the intermediate results to determine final results.

13. The system of claim 12, wherein the management computing system is further configured to:
obtain a set of sequence read data; and
create the set of shards by dividing the set of sequence read data into at least a number of subsets of sequence read data sized such that each subset of sequence read data fits within a data size limit associated with an instantiated serverless function computing device.

14. The system of claim 12, wherein each serverless function computing device is configured to:
retrieve a shard from the cloud storage system;
execute a serverless function on the shard; and
store an intermediate result in the cloud storage system.

15. The system of claim 12, wherein instantiating the number of serverless function computing devices includes instantiating a plurality of virtual machines in response to an instruction from the management computing system.

16. The system of claim 12, wherein the management computing system includes at least one computing device optimized for fast data storage and retrieval operations.

17. The system of claim 12, wherein the management computing system is provided by a cloud computing service.

18. The system of claim 17, wherein the cloud computing service provides a cloud-based virtual machine.

19. The system of claim 18, wherein a single platform provides the cloud-based virtual machine and at least one of the cloud storage system and the serverless function system.

20. The system of claim 12, wherein the management computing system includes at least one of a laptop computing device, a desktop computing device, a rack-mount server computing device, a mobile computing device, and one or more computing devices in a cloud computing system.
